(19) United States
(12) Patent Application Publication
(10) Pub. No.: US 2006/0008489 A1
(43) Pub. Date: Jan. 12, 2006

(54) Oily makeup cosmetics

(75) Inventors: Yuichiro Egawa, Kanagawa (JP); Noboru Hishikawa, Chiba (JP)

Correspondence Address: FOLEY AND LARDNER LLP;SUITE 500, 3000 K STREET NW, WASHINGTON, DC, 20007, US

(73) Assignees: KANEBO COSMETICS INC.; The Nisshin OilliO Group, Ltd.

(21) Appl. No.: 11/228,354

(22) Filed: Sep. 19, 2005

Related U.S. Application Data
(63) Continuation of application No. PCT/JP03/03295, filed on Mar. 19, 2003.

(30) Foreign Application Priority Data
Sep. 28, 2001 (JP) 2001-299639

(51) Int. Cl. A61K 8/37

(52) U.S. Cl. 424/401

(57) ABSTRACT

The present invention is to provide oily makeup cosmetics which comprises an oligo ester obtained from (a) an aliphatic acid or a hydroxy aliphatic acid having 8 to 30 carbon atoms (straight or branched, saturated or unsaturated), (b) a straight or branched dibasic acid having 12 to 36 carbon atoms, and (c) glycerol or a glycerol condensate, and a hydrocarbon having a branched structure.

### TECHNICAL FIELD

The present invention relates to oily makeup cosmetics, and is to provide oily makeup cosmetics which directs smooth feeling or excellent gloss at the time of coating, and excellent in stability with a lapse of time.

### BACKGROUND ART

Heretofore, it has widely been used to formulate a hydrocarbon having a branched structure such as polybutene, etc. to oily makeup cosmetics which are required to have glowing or gloss at finishing. For the purpose of solidifying or increasing a viscosity of the oily makeup cosmetics containing the hydrocarbon, it has been also well known to formulate solid wax or dextrin palmitate as a gelling agent. However, when the solid wax is to be formulated into oily makeup cosmetics required to provide glowing or gloss, if a formulation amount thereof is little, syneresis (sweating) tends to be caused, and there is a problem that glowing or gloss at finishing is impaired accompanied by increase in the formulation amount thereof. As used herein syneresis is the appearance of droplets on the surface of a composition that are visible to the naked eye. Also, dextrin palmitate is an oily gelling agent having an excellent gelling function to a variety oily basic agent, but it does not effectively act its gelling function onto polybutene, etc., and as a result, the resulting oily makeup cosmetics difficultly have a desired hardness or viscosity, and decrease in hardness or viscosity tends to be caused with a lapse of time, and at the worst case, there is a problem that the cosmetics are separated in composition. These problems can be solved with some extent by increasing a formulation amount of dextrin palmitate, but feeling becomes worse, and glowing or gloss is impaired as in the case where solid wax is formulated.

On the other hand, an oligo ester obtained from (a) an aliphatic acid or a hydroxy aliphatic acid each having 8 to 30 carbon atoms and each straight or branched, and saturated or unsaturated, (b) a straight or branched dibasic acid having 12 to 36 carbon atoms, (c) glycerol or a glycerol condensate is a conventionally known substance (Japanese Patent Publication No. Shou.61-7403, Japanese Unexamined Patent Publication No. Hei.7-126604), and it has also been reported that it is effective as an emulsifying aid for emulsified preparations (Japanese Unexamined Patent Publication No. 2000-219617). However, in oily makeup cosmetics comprising a hydrocarbon having a branched structure as an essential component, it has not been known that the conventional problems can be solved by formulating the same.

In such an actual circumstance, the present inventors have earnestly studied to solve the above-mentioned problems, and as a result, they have found that, in oily makeup cosmetics, by formulating an oligo ester obtained from (a) an aliphatic acid or a hydroxy aliphatic acid each having 8 to 30 carbon atoms and each straight or branched, and saturated or unsaturated, (b) a straight or branched dibasic acid having 12 to 36 carbon atoms, (c) glycerol or a glycerol condensate, and a hydrocarbon having a branched structure, oily makeup cosmetics having excellent in feeling in touch or stability with a lapse of time without impairing excellent gloss-providing effects possessed by the hydrocarbon having a branched structure can be obtained, whereby the present invention has been accomplished.

### SUMMARY OF THE INVENTION

That is, the present invention relates to oily makeup cosmetics which comprises an oligo ester obtained from (a) an aliphatic acid or a hydroxy aliphatic acid each having 8 to 30 carbon atoms and each straight or branched, and saturated or unsaturated, (b) a straight or branched dibasic acid having 12 to 36 carbon atoms and (c) glycerol or a glycerol condensate, and a hydrocarbon having a branched structure.

### BEST MODE FOR CARRYING OUT THE INVENTION

The oligo ester obtained from (a) an aliphatic acid or a hydroxy aliphatic acid each having 8 to 30 carbon atoms and each straight or branched, and saturated or unsaturated, (b) a straight or branched dibasic acid having 12 to 36 carbon atoms, and (c) glycerol or a glycerol condensate to be used in the present invention is a product which can be obtained by esterifying these materials according to the conventionally known method. As (a) the aliphatic acid or hydroxy aliphatic acid each having 8 to 30 carbon atoms and each straight or branched, saturated or unsaturated, there may be mentioned those in which the hydrocarbon group or hydroxy hydrocarbon group may be straight or branched, and may be saturated or unsaturated, for example, oleic acid, myristic acid, palmitic acid, lauric acid, stearic acid, behenic acid, isostearic acid, 12-hydroxystearic acid, octanoic acid, etc. As (b) the straight or branched dibasic acid having 12 to 36 carbon atoms, there may be mentioned, for example, eicosanoic diacid, 1,7-ethyloctadecane diacid, dodecane diacid, etc. As (c) the glycerol or the glycerol condensate, of these, glycerin condensate is a polyglycerin having a polymerization degree of 2 or more, preferably 2 to 10, and exemplified by diglycerin, triglycerin, tetraglycerin, hexaglycerin, decaglycerin, etc., and they can be used singly or as a mixture with glycerin. Of these oligo esters, particularly preferred is a compound represented by the following formula, in which (a) is behenic acid, (b) is eicosanoic diacid, (c) is glycerol or a glycerol condensate. They are commercially available as “NOMCORT HK-G”, “NOMCORT HK-P” from Nisshin Oillio Co., Ltd. and can be obtained easily. A formulation amount of the above-mentioned oligo ester in the present invention is suitable 0.5 to 10% by weight based on 100% by weight of the resulting oily makeup cosmetics. It is more preferably 1 to 8% by weight, and particularly preferably 2 to 5% by weight. If it is less than 0.5% by weight, effects of formulating a gelling agent are difficultly shown, while if it exceeds 10% by weight, feeling is markedly worsen and thus it is not preferred. Incidentally, the oily cosmetics in the present invention mean those which do not substantially contain water.

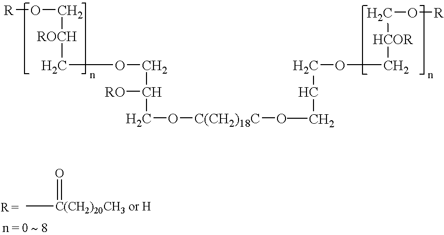

The hydrocarbon having a branched structure to be used in the present invention is a hydrocarbon having a structure in which the carbon chain is branched, and there may be mentioned an isoparaffin series hydrocarbon, or squalane, squalene, pristane, α-olefin oligomer, etc. The isoparaffin series hydrocarbon means a material containing isobutylene as a polymerization component, and there may be mentioned, for example, polybutene (polybutylene), light isoparaffin, light liquid isoparaffin (isoparaffin), heavy liquid isoparaffin (hydrogenated polybutene), polyisobutylene (butyl rubber), liquid isoparaffin (liquid polyisobutylene, hydrogenated polyisobutylene), etc. Incidentally, those in which the above materials are further purified by distillation can be suitable used. Of these, colorless liquid materials are preferred, isoparaffin series hydrocarbon is preferred in view of glossiness, and particularly preferably polybutene or heavy liquid isoparaffin (hydrogenated polybutene). Also, the number average molecular weight of the isoparaffin series hydrocarbon is preferably in the range of 500 to 2700. If the number average molecular weight is less than 500, there is a case where it is insufficient to provide glowing or gloss to oily makeup cosmetics at the time of coating, on the other hand, if it exceeds 2700, it is not preferred since there are cases where it becomes difficult to obtain oily makeup cosmetics which are preferred in the points of feeling such as extending property, stickiness, etc. Moreover, when handling at the time of preparing the oily makeup cosmetics is considered, the range of 800 to 1500 is more preferred. A formulation amount of the hydrocarbon having a branched structure in the present invention is suitably 5 to 80% by weight based on 100% by weight of the resulting oily makeup cosmetics. It is more preferably 15 to 50% by weight, and particularly preferably 20 to 40% by weight. If it is less than 5% by weight, there is a case where it is insufficient to provide glowing or gloss to oily makeup cosmetics at the time of coating, while if it exceeds 80% by weight, extending property, stickiness, etc. are markedly worsened.

In the oily makeup cosmetics of the present invention, other than the above-mentioned essential components, components of various kinds of powders, oily agents, UV ray inhibitors, fluorine compounds, resins, thickening agents, antiseptic agents, flavors, humectants, salts, solvents, antioxidants, chelating agents, insect inhibitors, etc., which are usually formulated in cosmetics may be used within the range which does not impair the effects of the present invention. Examples of the pigments to be used in the present invention are not limited in the shape (spherical, rod shaped, needle shaped, plate shaped, amorphous shaped, scaly shaped, spindle shaped, etc.) or particle size (fumed state, fine particulate, pigment class, etc.), particle structure (porous, non-porous, etc.) so long as it is used in a conventional cosmetics and any of them may be used, and there may be mentioned, for example, inorganic powder, organic powder, surfactant metal salt powder, colored pigment, pearl pigment, metal powder pigment, natural dye, etc., more specifically, as the inorganic powders, there may be mentioned pigment class titanium oxide, zirconium oxide, pigment class zinc oxide, cerium oxide, magnesium oxide, barium sulfate, calcium sulfate, magnesium sulfate, calcium carbonate, magnesium carbonate, talc, mica, kaolin, sericite, muscovite, synthetic mica, phlogopite, lepidolite, biotite, silicic acid, anhydrous silicic acid, aluminum silicate, magnesium silicate, aluminum magnesium silicate, calcium silicate, barium silicate, strontium silicate, tungstic acid metal salt, hydroxyapatite, vermiculite, Higilite™, bentonite, montmorillonite, hectorite, zeolite, ceramics powder, calcium secondary phosphate, alumina, aluminum hydroxide, boron nitride, silica, fine particulate titanium oxide, fine particulate zinc oxide, fine particulate cerium oxide, etc.; as the organic powders, there may be mentioned polyamide powder, polyester powder, polyethylene powder, polypropylene powder, polystyrene powder, polyurethane powder, benzoguanamine powder, polymethybenzoguanamine powder, polytetrafluoroethylene powder, Polymethyl methacrylate powder, cellulose, silk powder, Nylon powder, 12 Nylon, 6 Nylon, silicone powder, silicone rubber powder, silicone elastomer spherical powder, styrene-acrylic acid copolymer, divinylbenzene•styrene copolymer, vinyl resin, urea resin, phenol resin, fluorine resin, silicone resin, acrylic resin, melamine resin, epoxy resin, polycarbonate resin, microcrystalline fiber powder, starch powder, lauroyl lysine, etc.; as the surfactant metal salt powders (metallic soap), there may be mentioned zinc stearate, aluminum stearate, calcium stearate, magnesium stearate, zinc myristate, magnesium myristate, cetyl zinc phosphate, cetyl calcium phosphate, cetyl zinc sodium phosphate, etc.; as the colored pigments, there may be mentioned inorganic red pigments such as iron oxide, iron hydroxide and iron titanate, inorganic brown series pigments such as γ-iron oxide, etc., inorganic yellow series pigments such as yellow iron oxide, ocher, etc., inorganic black pigments such as black iron oxide, carbon black, etc., inorganic purple pigments such as manganese violet, cobalt violet, etc., inorganic green pigments such as chromium hydroxide, chromium oxide, cobalt oxide, cobalt titanate, etc., inorganic blue series pigments such as Prussian blue, ultramarine blue, etc., lake of tar series dyes, lake of natural dyes, synthetic resin powders in which these powders are complexed, etc.; as the pearl pigments, there may be mentioned titanium oxide-coated mica, bismuth oxychloride, titanium oxide-coated bismuth oxychloride, titanium oxide-coated talc, fish scale flake, titanium oxide-coated colored mica, etc.; as the tar dyes, there may be mentioned Red No. 104 (Pholxine B), Red No. 106 (Acid red), Red No. 201 (Lithol rubine B), Red No. 202 (Lithol rubine BCA), Red No. 218 (Tetrachlorotetrabromofluorescein), Red No. 223 (Tetrabromofluorescin, Red No. 226 (Helindone pink CN), Yellow No. 4 (Tartrazine), Yellow No. 5 (Sunset yellow FCF), Yellow No. 401 (Hanza yellow), Blue No. 1 (Brilliant blue FCF), Blue No. 404 (Phtalocyanine blue), Orange No. 201 (Dibromofluorescein), etc.; as the natural dyes, there may be mentioned powders selected from carminic acid, laccaic acid, carthamine, brazilin, crocin, etc., and as these powders, those in which the powders are complexed or treated with a general oily agents, silicone oils, fluorine compounds, surfactants, etc., may be used within the range which does not prevent the effects of the present invention. For example, they may be provisionally surface treated by a fluorine compound-treatment, silicone resin-treatment, pendant treatment, silane coupling agent-treatment, titan coupling agent-treatment, oily agent-treatment, polyacrylic acid-treatment, metallic soap-treatment, amino acid-treatment, inorganic compound-treatment, plasma treatment, mechanochemical treatment, etc., and depending on necessity, one kind or two or more kinds of the surface treatments may be used in combination.

Examples of the oily agents may include, for example, avocado oil, linseed oil, almond oil, insect (IBOTA) wax, perilla oil, olive oil, cacao butter, kapok wax, Torreya nucifera (kaya) oil, carnauba wax, cod liver oil, candelilla wax, beef tallow, beef foot fat, beef bone fat, hydrogenated beef tallow, apricot kernel oil, spermaceti, hydrogenated oil, wheat germ oil, sesame oil, rice germ oil, rice bran oil, corn wax, sasanqua oil, safflower oil, shea butter, Chinese tung oil, cinnamon oil, jojoba wax, shellac wax, turtle oil, soy bean oil, tea seed oil, camellia oil, evening primrose oil, corn oil, lard, rape seed oil, Japanese tung oil, rice bran wax, germ oil, horse oil, persic oil, palm oil, palm kernel oil, castor oil, hydrogenated castor oil, castor oil fatty acid methyl ester, sunflower oil, grape seed oil, bayberry wax, jojoba oil, macadamia nut oil, beeswax, mink oil, cottonseed oil, cotton wax, Japan wax, Japan kernel oil, montan wax, coconut oil, hydrogenated coconut oil, coconut fatty acid triglyceride, mutton-tallow, peanut oil, lanolin, liquid lanolin, reduced lanolin, lanolin alcohol, hard lanolin, lanolin acetate, lanolin fatty acid isopropyl ester, hexyl laurate, polyoxyethylene (hereinafter abbreviated to as POE) lanolin alcohol ether, POE lanolin alcohol acetate, lanolin fatty acid polyethylene glycol, POE hydrogenated lanolin alcohol ether, egg yolk oil, etc.; as the hydrocarbon oils, there may be mentioned ozokerite, ceresin, liquid paraffin, polyethylene wax, polyglyceryl monoisostearate, (adipic acid-2-ethylhexanoic acid•stearic acid) glyceryl oligo ester, (2-hexyldecanoic acid•sebacic acid) diglyceryl oligo ester, tri(caprylic•capric•myristic•stearic acid)glyceride, ethylene-propylene copolymer, microcrystalline wax, Vaseline, etc.; as the higher fatty acids, there may be mentioned lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, 12-hydroxystearic acid, etc.; as the higher alcohols, there may be mentioned lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, ceto stearyl alcohol, 2-decyl tetradecanol, cholesterol, phytosterol, POE cholesterol ether, monostearyl glycerin ether (batyl alcohol), monooleyl glyceryl ether (selachyl alcohol), etc.; as the ester oils, there may be mentioned diisobutyl adipate, 2-hexyldecyl adipate, di-2-heptylundecyl adipate, isononyl isononanate, isotridecyl isononanate, N-alkylglycol monoisostearate, isocetyl isostearate, trimethylolpropane triisostearate, neopentyl glycol di-2-ethylhexanoate, cetyl 2-ethylhexanoate, trimethylolpropane tri-2-ethylhexanoate, polyglyceryl diisostearate, polyglyceryl triisostearate, polyglyceryl tetraisostearate, pentaerythritol tetra-2-ethylhexanoate, cetyl octanoate, octyldodecyl rubber ester, oleyl oleate, octyldodecyl oleate, decyl oleate, phytosteryl oleate, neopentyl glycol dicaprate, 2-ethylhexyl succinate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, octyl palmitate, cholesteryl 12-hydroxystearate, dipentaerythritol fatty acid ester, isopropyl myristate, octyldodecyl myristate, isostearyl myristate, 2-hexyldecyl myristate, myristyl myristate, hexyldecyl dimethyloctanoate, ethyl laurate, hexyl laurate, di(cholesteryl•octyldodecyl) N-lauroyl-L-glutamate, di(cholesteryl•behenyl•octyldodecyl) N-lauroyl-L-glutamate, di(phytosteryl•octyldodecyl) N-lauroyl-L-glutamate, di(phytosteryl•behenyl•octyldodecyl) N-lauroyl-L-glutamate, diisostearyl malate, glyceryl triisooctanoate, tri(capryl•capric acid)glycerin, glyceryl triisostearate, glyceryl triisopalmitate, glyceryl monostearate, glyceryl di-2-heptylundecanoate, glyceryl trimyristate, diglyceryl myristate isostearate, etc.

Also, examples of the other form of oily agents, there may be mentioned, for example, silicone compounds such as dimethylpolysiloxane, methyl hydrogen polysiloxane, methylphenyl polysiloxane, alkyl-modified organopoly-siloxane, terminal-modified organopolysiloxane, fluorine-modified organopolysiloxane, amino-modified organopoly-siloxane, polyether-modified silicone, perfluoroalkyl•poly-oxyalkylene co-modified organopolysiloxane, acryl-modified silicone, glyceryl-modified silicone, polyglyceryl-modified silicone, sugar-modified silicone, silicone gel, silicone RTV rubber, etc., fluorine compounds such as perfluoropolyether, fluorinated pitch, fluorocarbon, fluoro alcohol, etc.

As an ultraviolet ray-inhibiting component, there may be mentioned inorganic series and organic series ultraviolet ray inhibiting agents. Examples of the inorganic series ones, there may be mentioned, for example, metal oxides such as titanium dioxide, titanium low-level oxide, zinc oxide, cerium oxide, etc., metal hydroxides such as iron hydroxide, etc., metal flakes such as plate iron oxide, aluminum flake, etc., ceramics such as silicon carbide, etc. Of these, it is particularly preferred that the material is at least one selected from fine particulate metal oxide and fine particulate metal hydroxide each having an average particle size of 5 to 100 nm. These powders are preferably subjected to surface treatment by the conventionally known surface treatment, for example, fluorine compound treatment (perfluoroalkylphosphoric acid ester treatment, perfluoroalkylsilane treatment, perfluoropolyether treatment, fluorosilicone treatment, fluorinated silicone resin treatment is preferred), silicone treatment (methylhydrogenpolysiloxane treatment, dimethylpolysiloxane treatment, vapor phase method tetramethyltetrahydrogen-cyclotetrasiloxane treatment is preferred), silicone resin treatment (trimethylsiloxysilicic acid treatment is preferred), pendant treatment (a method of adding alkyl chain, etc., after vapor phase method-silicone treatment), silane coupling agent treatment, titanium coupling agent treatment, silane treatment (alkylsilane or alkylsilazane treatment is preferred), oily agent treatment, N-allylated lysine treatment, polyacrylic acid treatment, metal soap treatment (stearic acid or myristic acid salt is preferred), acryl resin treatment, metal oxide treatment, etc., more preferably, these treatments are carried out by combining a plural kind thereof. For example, there may be mentioned a method in which fine particulate titanium oxide surface is coated with silicon oxide or a metal oxide such as alumina, etc., and then subjected to surface treatment with alkyl silane and the like. An amount of the surface treatment is preferably within the range of 0.1 to 50% by weight in a total amount of the surface treatment based on the weight of the powder.

Also, examples of the organic series UV ray inhibitors, there may be mentioned, for example, glyceryl mono-octanoate di-p-methoxy cinnamate, 2-ethylhexyl p-methoxy cinnamate (another name; octyl p-methoxy cinnamate), 2-hydroxy-4-methoxybenzophenone, 2-hydroxy-4-methoxybenzo-phenone-5-sulfuric acid, 2,2′-dihydroxy-4-methoxybenzo-phenone, p-methoxyhydrocinnamate diethanolamine salt, p-aminobenzoic acid (hereinafter abbreviated to as PABA), ethyldihydroxypropyl PABA, glyceryl PABA, homomenthyl salicylate, methyl-O-aminobenzoate, 2-ethylhexyl-2-cyano-3,3-diphenylacrylate, octyl dimethyl PABA, octyl salicylate, 2-phenyl-benzimidazol-5-sulfate, triethanolamine salicylate, 3-(4-methylbenzylidene)camphor, 2,4-dihydroxybenzophenone, 2,2′,4,4′-tetrahydroxybenzophenone, 2,2′-dihydroxy-4,4′-dimethoxybenzophenone, 2-hydroxy-4-N-octoxy-benzophenone, 4-isopropyl dibenzoylmethane, butylmethoxy-dibenzoylmethane, octyltriazone, 4-(3,4-dimethoxyphenyl-methylene)-2,5-dioxo-1-imidazolidine 2-ethylhexyl propionate, and polymer derivatives and silane derivatives of the above-mentioned compounds, etc. Also, it is possible to use those in which the organic series UV ray inhibitors are encapsulated into polymer powder. The polymer powder may be hollow or not may be hollow, and as an average primary particle size may be within the range of 0.1 to 50 μm, and the particle size distribution may be broad or may be shape. As a kind of the polymer, there may be mentioned an acrylic resin, a methacrylic resin, a styrene resin, a polyurethane resin, polyethylene, polypropylene, polyethylene terephthalate, a silicone resin, Nylon, an acrylamide resin, etc. Preferred are powder in which organic series UV ray inhibitors are incorporated in these polymer powders in an amount in the range of 0.1 to 30% by weight based on the weight of the powder, particularly preferably 4-tert-butyl-4′-methoxydibenzoylmethane which is a UVA absorbent is formulated. Among the above-mentioned UV ray inhibitor components, it is preferred to use at least one selected from the group consisting of fine particulate titanium oxide, fine particulate zinc oxide, 2-ethylhexyl paramethoxycinnamate, butylmethoxydibenzoylmethane, oxybenzone and benzophenone series UV ray absorbent is generally used, commercially available, and has high UV ray inhibiting effects. In particular, it is preferred to use the inorganic series and organic series ones in combination. Also, it is suitable to use that corresponding to UV-A and that corresponding to UV-B in combination.

Examples of the thickening agents may include vegetable series polymers such as acacia, tragacanth, arabinogalactan, locust bean gum (carob gum), guar gum, karaya gum, carrageenan, pectin, agar, quince seed (quince), starch (rice, corn, potato, wheat), algae colloid, gum tragacanth, locust bean gum, etc., microorganism series polymers such as xanthan gum, dextran, succinoglucan, pullulan, etc., animal series polymers such as collagen, casein, albumin, etc., starch series polymers such as carboxymethyl starch, methylhydroxypropyl starch, etc., cellulose series polymers such as methyl cellulose, ethyl cellulose, methylhydroxypropyl cellulose, carboxymethyl cellulose, hydroxymethyl cellulose, hydroxypropyl cellulose, nitro cellulose, sodium sulfate cellulose, sodium carboxymethyl cellulose, crystalline cellulose, cellulose powder, etc., alginic acid series polymers such as sodium alginate, alginic acid propylene glycol ester, etc., vinyl series polymers such as polyvinylmethyl ether, polyvinylpyrrolidone, carboxyvinyl polymer, etc., polyoxyethylene series polymers such as polyethylene glycol, etc., acrylic series polymers such as polyoxyethylene polyoxypropylene copolymer series polymer, sodium polyacrylate, polyethyl acrylate, polyacrylic acid amide, etc., polyethyleneimine, cationic polymer, inorganic series thickening agents such as bentonite, aluminum magnesium silicate, laponite, smectite, saponite, hectorite, anhydrous silicic acid, etc. Also, as the other thickening agents, there is an oil-soluble gelling agent, and may be mentioned, for example, metal soaps such as aluminum stearate, magnesium stearate, zinc myristate, etc., amino acid derivatives such as N-lauroyl-L-glutamic acid, α,γ-di-n-butylamine, etc., dextrin fatty acid esters such as dextrin palmitic acid ester, dextrin stearic acid ester, dextrin 2-ethylhexanoic acid palmitic acid ester, etc., sucrose fatty acid esters such as sucrose palmitic acid ester, sucrose stearic acid ester, etc., benzylidene derivatives of sorbitol such as monobenzylidene sorbitol, dibenzylidene sorbitol, etc., organic modified clay minerals such as dimethylbenzyldodecyl ammonium montmorillonite clay, dimethyldioctadecyl ammonium montmorillonite, octadecyldimethylbenzyl ammonium montmorillonite, etc.

As the physiologically active components to be used in the present invention, substances which provide any physiological activity to skin when it is coated onto skin may be mentioned. For example, there may be mentioned anti-inflammatory agents, anti-aging agents, astringents, humectants, blood circulation promoters, antibacterial agents, bactericides, dryers, cooling agents, warming agents, vitamins, amino acids, wound healing promoters, torpents, analgesics, cell activating agents, enzyme components, etc. Of these, natural series vegetable extracted components, seaweed extracted components, crude drug components are particularly preferred. In the present invention, one kind or two or more kinds of these physiologically active components is/are preferably formulated. For example, there may be mentioned angelica extract, locust (acacia) extract, silvervine extract, avocado extract, hydrangea extract, althea extract, arnica extract, aloe extract, apricot extract, apricot kernel extract, ginkgo extract, turmeric extract, woolong tea extract, rose fruit extract, Echinacea leaf extract, Scutellaria root extract, phellodendron bark extract, barley extract, horse chestnut extract, leek extract, hollyhock extract, silvermound artemisia extract, sea-bindweed extract, green tea extract, marguerite extract, cinnamon extract, tear grass extract, Hypericum extract, white nettle extract, watercress extract, orange extract, saffron extract, loquat extract, sea salt, hydrolyzed elastin, hydrolyzed wheat powder, hydrolyzed silk, cassis extract, sun spurge extract, fennel extract, golden bells extract, gentian extract, chamomile extract, carrot extract, Artemisia capillaris extract, hibiscus extract, kiwi extract, geranium herb extract, ganoderma extract, cinchona extract, cucumber extract, guanosine, gardenia extract, hotarukazura (Lithospermum zollingeri) extract, lemon balm extract, olive extract, Oenothera erythrosepara extract, Sasa Albomarginata extract, sophora extract, walnut extract, grape fruit extract, plantain extract, Japanese knotweed extract, Sambucus nigra extract, clematis extract, Rhei rhizoma extract, great duckweed extract, chickweed extract, clove extract, nettle extract, thyme extract, chlorella extract, mulberry bark extract, gentian extract, black tea extract, yeast extract, burdock root extract, fermented rice bran extract, rice germ oil, comfrey extract, collagen, bilberry extract, Asiasarum root extract, Bupleuri radix extract, umbilical cord extract, salvia extract, Saponaria extract, bamboo grass extract, Crataegus Fruit extract, Zanthoxylum fruit extract, shiitake extract, Rehmannia root extract, Lithospermum root extract, perilla herb extract, linden extract, Filipendula extract, peony root extract, Calamus rhizome extract, birch extract, horsetail extract, ivy extract, Crataegus extract, Sambucus extract, yarrow extract, peppermint extract, sage extract, mallow extract, Cnidium rhizome extract, Swertia herb extract, soy extract, jujube extract, thyme extract, cogon grass extract, citrus unshiu peel extract, Japanes angelica root extract, calendula extract, perch seed extract, bitter orange peel extract, Houttuynia extract, tomato extract, Natto extract, ginseng extract, garlic extract, wild rose extract, hibiscus extract, Ophiopogon extract, lotus extract, parsley extract, honey, pellitory extract, Isodonis extract, bisabolol, coltsfoot extract, butterbur scape extract, hoelen extract, Butcher broom extract, grape extract, propolis, sponge gourd extract, safflower extract, peppermint extract, linden extract, Paeonia extract, hop extract, pine extract, skunk cabbage extract, Mukurossi peel extract, peach extract, cornflower extract, eucalyptus extract, saxifrage extract, yuzu (Citrus junos) extract, coix extract, mugwort extract, lavender extract, lettuce extract, lemon extract, Chinese milk vetch extract, rose extract, romanchamomile extract, royal jelly extract, etc.

Also, there may be mentioned biopolymers such as mucopolysaccharide, sodium hyaluronate, chondroitin sodium sulfate, collagen, elastin, chitin, chitosan, hydrolyzed egg shell membrane, etc., amino acids such as glycine, valine, leucine, isoleucine, serine, threonine, phenylalanine, alginine, lysine, aspartic acid, glutamic acid, cystine, cysteine, methionine, triptophane, etc., hormones such as estradiol, ethenyl estradiol, etc., humectants such as amino acid, sodium lactate, urea, sodium pyrrolidone carboxylate, betaine, whey, etc., oily components such as sphingolipids, ceramide, cholesterol, cholesterol derivatives, phospholipids, etc., anti-inflammatory agents such as ε-aminocaprioc acid, glycyrrhizic acid, β-glycyrehtinic acid, lysozyme chloride, guaiazulene, hydrocortisone, allantoin, tranexamic acid, azulene, etc., active components such as Vitamins A, B2, B6, C, D, K, vitamin C glycosides, calcium pantothenate, biotin, nicotinic acid amide, allantoin, diisopropylamine dichloroacetate, 4-aminomethylcyclohexane carboxylic acid, etc., cell activators such as γ-hydroxy acid, β-hydroxy acid, etc., blood circulation promoters such as γ-oryzanol, etc., wound healing agents such as retinol, retinol derivatives, etc., refrigerants such as cepharanthine, glycyrrhiza extract, capsicum tincture, hinokitiol, iodized garlic extract, pyridoxine hydrochloride, nicotinic acid, nicotinic acid derivatives, calcium pantothenate, D-pantothenyl alcohol, acetyl pantothenyl ethyl ether, biotin, allantoin, iso-propylmethylphenol, estradiol, ethenyl estradiol, capronium chloride, benzalkonium chloride, diphenhydramine hydrochloride, takanal, camphor, salicylic acid, nonylic vanillyl amide, nonanoic acid vanillylamide, piroctone olamine, glyceryl pentadecanoate, 1-menthol, etc.

As oily makeup cosmetics of the present invention, there may be mentioned lipstick, lip gloss, lip cream, foundation, concealer, eye shadow, eye gloss, eyeliner, eyebrow, etc. Moreover, a shape of the products is not specifically limited and may be applied to a liquid state, a stick state, a solid state, a paste state, etc.

### EXAMPLES

In the following, the present invention is explained in more detail by referring to Examples and Comparative examples. Incidentally, as the (behenic acid/eicosanoic diacid)glyceryl in the following prescription, “NOMUCORT HK-G” available from Nisshin Oillio was used.

Evaluation methods with regard to various characteristics of the respective oily makeup cosmetics in Example and Comparative example are shown below.

[Evaluation of Skin Availability]

Each 20 member of special panelists were prepared (provided that panelists are sometimes overlapped with the items to be tested) with regard to the respective evaluation items, and from the number of the panelists judged as excellent in the respective evaluation items, evaluation was carried out according to the classifications mentioned below.

| Number of persons answered | |
|---|---|
| to “good” among 20 persons | Evaluation |
| 15 persons or more | ⊚ |
| 10 to 14 persons | ◯ |
| 5 to 9 persons | Δ |
| 0 to 4 persons | X |

[Hardness]

Oily makeup cosmetics were degassed and then filled in an ointment bottle with a volume of 10 mL at 100° C., solidified by allowing to stand for cooling, allowed to stand in a thermostat at 30° C. for 6 hours or longer, and a peak value measured by RHEOMETER (RHEO TEX manufactured by Sun Scientific Co., Ltd., plunger diameter: 10 mmφ, rest elevating rate: 1 mm/sec) was made a hardness.

[Stability]

Oily makeup cosmetics were charged in respective thermostat chambers at 5° C., 30° C. and 45° C., for 3 months and observed whether any change in quality occurred or not, and judged when absolutely no problem occurred as ◯, that which can observe any sigh causing abnormality as Δ, and that caused abnormality as ×.

### Example 1

### Comparative Examples 1 to 2 (Solid Oily Lipstick of Flow-In Type)

In the composition (numerals are % by weight) shown below:
Polybutene 25.0
Diisostearyl malate 25.0
2-Ethylhexyl hydroxystearate remainder
Liquid paraffin 5.0
Isotridecyl isononanate 3.0
Red No. 202 (Lithol rubine BCA) 0.4
Titanium oxide 1.0
Red ocher-coated titanium-mica 5.0

investigation was carried out whether how many amounts of (behenic acid/eicosanoic diacid)glyceryl, ceresin and dextrin palmitate are added to the solid oily lipstick as gelling agents so that a degree of caught to a lip brush or fingers becomes optimum (around 200 g according to the above-mentioned hardness measurement method) as the solid oily lipstick, and according to the results thereof, solid oily lipsticks shown in Table 1 were prepared according to the conventional manner, then, they were each poured into a resin-made compact vessel, and various kinds of evaluations were carried out. The evaluation results are also shown in Table 1.

TABLE 1

| | Example | Comparative example | |
|---|---|---|---|
| | 1 | 1 | 2 |
| (Behenic acid/eicosanoic diacid)- | 4.0 | | |
| glyceryl | | | |
| Cerecin | | 8.0 | |
| Dextrin palmitate | | | 12.0 |
| Polybutene | 25.0 | 25.0 | 25.0 |
| Diisostearyl malate | 25.0 | 25.0 | 25.0 |
| 2-Ethylhexyl hydroxystearate | Remainder | Remainder | Remainder |
| Liquid paraffin | 5.0 | 5.0 | 5.0 |
| Isotridecyl isononanate | 3.0 | 3.0 | 3.0 |
| Red No. 202(Lithol rubine BCA) | 0.4 | 0.4 | 0.4 |
| Titanium oxide | 1.0 | 1.0 | 1.0 |
| Red ocher-coated titanium-mica | 5.0 | 5.0 | 5.0 |
| Gloss at the time of coating | ⊚ | ◯ | Δ |
| Feeling | ⊚ | Δ | X |
| Storage stability | ◯ | X (※) | ◯ |
| Hardness | 210 | 200 | 200 |

※: sweating at 30° C. and 45° C.

As can be clearly seen from the results in Table 1, lipstick of the present invention was excellent in gloss and feeling at the time of coating and storage stability.

### Examples 2 to 3

### Comparative Examples 3 to 5 (Liquid Lip Gloss)

According to the prescription shown in Table 2, liquid lip glosses were prepared according to the conventional manner, and then filled in a bottle vessel with a lip brush, and various kinds of evaluations were carried out. The evaluation results are also shown in Table 2.

TABLE 2

| | Example | | Comparative example | | |
|---|---|---|---|---|---|
| | 2 | 3 | 2 | 3 | 4 |
| Glyceryl(behenic | 0.5 | 2.0 | | | |
| acid/eicosanoic diacid) | | | | | |
| Dextrin palmitate | | | 5.0 | 5.0 | |
| Hydrogenated polybutene | 80.0 | 40.0 | 80.0 | 40.0 | 90.0 |
| Diisostearyl malate | | 20.0 | | 20.0 | |
| 2-Ethylhexyl hydroxystearate | Remainder | Remainder | Remainder | Remainder | Remainder |
| Methyl phenyl polysiloxane | | 3.0 | | 3.0 | |
| α-Olefin oligomer | 5.0 | 5.0 | 5.0 | 5.0 | |
| Lauroyl lysine-treated | 0.1 | 0.1 | 0.1 | 0.1 | 0.1 |
| Red No. 201 (Lithol | | | | | |
| rubine B) | | | | | |
| Lauroyl lysine-treated | 0.1 | 0.1 | 0.1 | 0.1 | 0.1 |
| Yellow No. 4 (Tartrazine) | | | | | |
| Lauroyl lysine-treated | 2.0 | 2.0 | 2.0 | 2.0 | 2.0 |
| titanium-mica | | | | | |
| Gloss at the time of | ⊚ | ⊚ | ◯ | Δ | ⊚ |
| coating | | | | | |
| Feeling | ◯ | ⊚ | Δ | ◯ | X |
| Storage stability | ◯ | ◯ | X (※1) | Δ (※2) | X |

※1: Separated and pigment precipitated at 45° C.
※2: Seems to be separated at 45° C.

As can be clearly seen from the results in Table 2, lip glosses of the present invention were excellent in gloss and feeling at the time of coating and storage stability.

### Example 4

### Solid Eye Gloss of Flow-In Type

Glyceryl (behenic acid/eicosanoic diacid) 10.0
Microcrystalline wax 2.0
Dextrin palmitate 2.0
Polybutene 5.0
Glyceryl triocanoate remainder
Liquid paraffin 20.0
Jojoba oil 10.0
Propylene glycol dicaprylate 10.0
Isononyl isononanate 5.0
Methylpolysiloxane 10.0
Silica beads 10.0
Polyalkyl acrylate 3.0
Red No. 226 (Helindone pink CN) 0.1
(PET/Al/epoxy resin) laminated powder 5.0

Eye gloss of Example 4 was excellent in gloss and feeling at the time of coating and storage stability.

### Example 5

### Lipstick

Titanium-mica 3.0
Aluminum lake of Red No. 104(1) 0.4
(Aluminum lake of phloxine B)
Aluminum lake of Blue No. 1 0.5
(Aluminum lake of brilliant blue FCF)
Orange No. 201 0.2
(Dibromofluorescein)
Ceresin 8.0
Microcrystalline wax 5.0
Paraffin 4.0
Hydrogenated polybutene 25.0
Cetyl isooctanoate 2.0
Glyceryl (behenate/eicosanoic diacid) 5.0
Vegetable squalane 0.5
Natural vitamin E 0.5
Octyl oxystearate 15.0
octyldodecanol 5.0
Tri(caprylic-capric-myristic-stearic acid)glyceride 5.0
Tri (caprylic-capric acid) glycerin 10.0
Octyldodecyl ricinoleate 10.9

### Example 6

### Foundation

Candelilla wax 2.0
Carnauba wax 1.0
Glyceryl (behenate/eicosanoic diacid) 0.5
Polyethyleneglycol monostearate (45E.O.) 0.6
methylpolysiloxane (20cs) 6.0
Diglycerin oleate 1.5
Liquid isoparaffin (available from Nippon Oil & Fats Co.,
Ltd., Parleam 6) 15.0
Polybutene 5.0
2-Ethylhexyl p-dimethylbenzoate 1.0
Isopropyl isostearate remainder
Anhydrous silicic acid 0.5
Polyethylene powder 1.0
Titanium oxide 16.0
Polyethylene-treated Red ocher 0.8
Polyethylene-treated yellow iron oxide 3.5
Polyethylene-treated black iron oxide 0.3
Polyethylene-treated kaolin 10.0
Nylon powder 3.0
Poly(alkyl acrylate) 10.0

### UTRILIZABILITY IN INDUSTRY

As explained in detail above, it is clear that the oily makeup cosmetics of the present invention are excellent in gloss and feeling at the time of coating and storage stability.

1. Oily makeup cosmetics comprising an oligo ester obtained from the following components, and a hydrocarbon having a branched structure.
(a) an aliphatic acid or a hydroxy aliphatic acid each having 8 to 30 carbon atoms and each straight or branched, and saturated or unsaturated,
(b) a straight or branched dibasic acid having 12 to 36 carbon atoms,
(c) glycerol or a glycerol condensate

2. The oily makeup cosmetics according to claim 1, wherein a content of the oligo ester is 0.5 to 10% by weight, and a content of the hydrocarbon having a branched structure is 5 to 80% by weight.

3. The oily makeup cosmetics according to claim 2, wherein the hydrocarbon having a branched structure is an isoparaffin series hydrocarbon.

4. The oily makeup cosmetics according to claim 2, wherein the hydrocarbon having a branched structure is a polybutene or a hydrogenated polybutene.

5. The oily makeup cosmetics according to claim 1, wherein the oligo ester comprises the following components.
(a) behenic acid
(b) eicosanoic diacid
(c) glycerol or a glycerol condensate

6. The oily makeup cosmetics according to claim 2, wherein the oligo ester comprises the following components.
(a) behenic acid
(b) eicosanoic diacid
(c) glycerol or a glycerol condensate

7. The oily makeup cosmetics according to claim 3, wherein the oligo ester comprises the following components.
(a) behenic acid
(b) eicosanoic diacid
(c) glycerol or a glycerol condensate

8. The oily makeup cosmetics according to claim 4, wherein the oligo ester comprises the following components.
(a) behenic acid
(b) eicosanoic diacid
(c) glycerol or a glycerol condensate

9. The oily makeup cosmetics according to claim 2, wherein the hydrocarbon having a branched structure is an iso-paraffin series hydrocarbon having a number average molecular weight of 500 to 2700.
